(19) United States
(12) Patent Application Publication
(10) Pub. No.: US 2022/0347220 A1
(43) Pub. Date: Nov. 3, 2022

(54) FORMULATIONS AND METHODS FOR ACTIVATING SIGNALING MOLECULES OUTSIDE A NATIVE PHYSIOLOGICAL RESPONSE

(71) Applicant: Brian Liljenquist, Salt Lake City, UT (US)

(72) Inventor: Brian Liljenquist, Salt Lake City, UT (US)

(21) Appl. No.: 17/242,575

(22) Filed: Apr. 28, 2021

(51) Int. Cl. A61K 35/19; A61K 35/16; A61K 38/36; A61K 35/50; A61K 35/28; A61K 35/35

(52) U.S. Cl. CPC A61K 35/19; A61K 35/16; A61K 38/363; A61K 35/50; A61K 35/28; A61K 35/35

(57) ABSTRACT

Formulations and methods for activating signaling molecules outside a native physiological response are provided. The formulations can include an activator component and a signaling component containing both signaling molecules and mesenchymal stem cells. When a formulation containing both an activator component and a signaling component is injected into or applied to a treatment area, the activator component may activate the signaling molecules of the signaling component which in turn signal the mesenchymal stem cells of the signaling component to regrow, repair or otherwise provide a benefit to the treatment area.

### CROSS-REFERENCE TO RELATED APPLICATIONS

N/A

### BACKGROUND

Over the past decade, platelet-rich plasma (PRP) has been extensively studied as a treatment for structural disorders such as osteoarthritis, tendinopathy and acute muscle damage as well as for other disorders. It is believed that PRP may treat such disorders by releasing growth factors and other molecules that play a role in various tissue healing processes such as inflammatory modulation, chondrocyte apoptosis inhibition, bone and vessel remodeling and collagen synthesis. PRP may also release fibrin which may act as a scaffold and chemo-attractant for other cells that play a role in the healing response.

Amniotic fluid (AF) has also been studied as a treatment for skin and eye disorders and more recently for joint and other structural disorders. As with PRP, it is believed that AF may treat such disorders by releasing growth factors and other molecules that play a role in various tissue healing processes.

When either PRP or AF is used to treat a disorder, such as by injecting PRP or AF into a joint, there is typically a noticeable response, such as a reduction in pain or swelling, that lasts from days to weeks. After this short-lived response, the disorder typically returns to pre-treatment conditions. Therefore, to receive adequate treatment for such disorders, patients oftentimes need to return to their physicians frequently to receive follow-up treatments of PRP or AF.

### BRIEF SUMMARY

Embodiments of the present invention extend to formulations and methods for activating signaling molecules outside a native physiological response. The formulations can include an activator component and a signaling component containing both signaling molecules and mesenchymal stem cells. When a formulation containing both an activator component and a signaling component is injected into or applied to a treatment area, the activator component may activate the signaling molecules of the signaling component which in turn signal the mesenchymal stem cells of the signaling component to regrow, repair or otherwise provide a benefit to the treatment area.

In some embodiments, the present invention may be implemented as a formulation for activating signaling molecules outside a native physiological response. The formulation may include a first volume of an activator component. The formulation may also include a second volume of a signaling component.

In some embodiments, the present invention may be implemented as a method for activating signaling molecules outside a native physiological response. A formulation, which includes a first volume of an activator component and a second volume of a signaling component, can be obtained. The formulation can then be injected or applied to a treatment area of a patient.

In some embodiments, the present invention may be implemented as a formulation for activating signaling molecules outside a native physiological response. The formulation may include an activator component comprising one or more of platelet-poor plasma, platelet-rich plasma or platelet-rich fibrinogen. The formulation may also include a signaling component comprising one or more of amniotic fluid or exosomes isolated from bone marrow or fat.

This summary is provided to introduce a selection of concepts in a simplified form that are further described below in the Detailed Description. This Summary is not intended to identify key features or essential features of the claimed subject matter.

### DETAILED DESCRIPTION

For purposes of this description and the claims, the following terms shall be construed as defined.

“Effective amount” refers to an amount of a formulation that may be injected into or applied to a treatment area of a patient to alleviate, delay onset of, or prevent one or more symptoms, particularly of a disease or disorder described herein.

“Patient” refers to a human or an animal.

“Treatment area” means any area of a patient's body where a formulation may be injected or applied.

“Native physiological response” refers to a physiological response that naturally occurs within a patient's body.

“Signaling molecules” refer to molecules that can bind to cell receptors to initiate a physiological response.

“Activating signaling molecules” means causing signaling molecules to bind to cell receptors to initiate a physiological response.

Formulations and methods for activating signaling molecules outside a native physiological response are provided. The formulations can include an activator component and a signaling component containing both signaling molecules and mesenchymal stem cells. When a formulation containing both an activator component and a signaling component is injected into or applied to a treatment area, the activator component may activate the signaling molecules of the signaling component which in turn signal the mesenchymal stem cells of the signaling component to regrow, repair or otherwise provide a benefit to the treatment area.

The activator component of some formulations may be platelet-poor plasma (PPP), platelet-rich plasma (PRP), platelet-rich fibrinogen (PRF), whole blood, partial blood or other component(s) of blood. In some embodiments, the activator component may include only one of PPP, PRP or PRF. In other embodiments, the activator component may include more than one or all of PPP, PRP and PRF. The signaling component of some formulations may be amniotic fluid (AF) or exosomes isolated from bone marrow or fat. The activator component and the signaling component may be derived from humans or from any of a variety of other species such as porcine and bovine species.

The formulations may include a first volume of the activator component and a second volume of the signaling component. In some embodiments, the first volume may be greater than the second volume. In some embodiments, the first volume may be between 50% and 150% greater than the second volume. For example, an effective amount of the formulation may include 3-5 milliliters of the activator component and 2 milliliters of the signaling component. In other embodiments, the first volume may be less than the second volume or equal to the second volume.

In some embodiments, the activator component may be prepared from whole blood, including from a patient's whole blood. For example, a blood sample may be collected from a patient or another individual or animal and spun in a centrifuge to separate the activator component from red blood cells. In some embodiments, such as when the activator component is PPP or PRP, to minimize the risk of the activator component clotting when the formulation is injected, the blood sample may be centrifuged to maximize the separation of the red blood cells, e.g., to minimize the hematocrit of the resulting PPP or PRP. Through testing, it does not appear that an activator component having low hematocrit decreases the efficacy of the formulation even though the low hematocrit activator component would, in theory, have fewer growth factors and other signaling components. Also, in some embodiments, prior to centrifuging the blood sample to produce the activator component, an anticoagulant such as heparin may be added to the blood sample to further minimize the risk that the activator component may clot when the formulation is injected.

In contrast to PPP and PRP, PRF may have (or be prepared to have) a high hematocrit and therefore may readily clot when the formulation is injected. Accordingly, formulations that employ PRF as the activator component may be most suitable for use in treatment areas where a natural filler is desired in addition to the activation of signaling molecules outside a native physiological response.

Whether or not the activator component is processed to minimize the likelihood of clotting, a first volume of the activator component can be mixed with a second volume of the signaling component to produce the formulation. In some embodiments, the formulation may consist only of the activator component and the signaling component. In other embodiments, the formulation may include one or more other components in addition to the activator component and the signaling component. Also, the formulation may be injected immediately upon mixing the activator component and the signaling component or may be refrigerated or frozen.

Although the exact mechanisms by which such formulations work are still being studied, it is believed that, when the formulations are injected into a treatment area, signaling molecules or other elements in the activator component activate signaling molecules in the signaling component which in turn signal the mesenchymal stem cells in the signaling component to regrow, repair or otherwise provide a benefit to the treatment area. Notably, although PRP and AF individually have shown some efficacy as injectable treatments for various disorders, research and testing has shown that formulations containing both PRP and AF or another activator component/signaling component combination exhibit synergistic benefits. For example, in comparison to the days-long or possibly weeks-long improvements that PRP and AF individually may provide, formulations configured in accordance with embodiments of the present invention have provided clinically remarkable results that last for months. As a potential explanation for this synergy, it is believed that, once the formulation is injected into or applied to a treatment area, the activator component may activate the signaling molecules in the signaling component with far greater efficacy than any other mechanism that may naturally exist or occur at the treatment area. This includes any mechanism that may be invoked when PRP or AF is individually injected.

Formulations containing an activator component and a signaling component in accordance with embodiments of the present invention may be used in a wide variety of clinical applications on both human and non-human patients. The following are some examples and should not be viewed as limiting how such formulations may be used.

In some embodiments, the formulation may be used as a subcutaneous injection into the scalp for hair restoration.

In some embodiments, the formulation may be used as a subcutaneous injection in and around the face to encourage an inflammatory response to repair damaged areas of skin.

In some embodiments, the formulation may be applied through microneedling to any portion of the skin to treat autoimmune reactions such as vitiligo, to perform scar revision or stretch mark repair, to rejuvenate skin or wrinkles, etc.

In some embodiments, the formulation may be used as a subcutaneous injection to function as a natural biological filler anywhere in the body. In such embodiments, the formulation may preferably use PRF as the activator component given its propensity to form a clot that can enhance the filling effect.

In some embodiments, the formulation may be used as an intravaginal injection for vaginal rejuvenation.

In some embodiments, the formulation may be injected into the musculature of the penis to increase volume, sensitivity and function.

In some embodiments, the formulation may be used as an intra-articular injection in any joint to increase intra-articular fluid volume or lubrication of the joint, to decrease inflammation, to encourage repair and regrowth of articular surface defects, to repair intra-articular and/or extra-articular ligaments, etc.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

1. A formulation for activating signaling molecules outside a native physiological response comprising:
a first volume of an activator component; and
a second volume of a signaling component.

2. The formulation of claim 1, wherein the activator component is one of:
platelet-poor plasma;
platelet-rich plasma; or
platelet-rich fibrinogen.

3. The formulation of claim 1, wherein the signaling component is one of:
amniotic fluid; or
exosomes isolated from bone marrow or fat.

4. The formulation of claim 1, wherein the activator component is platelet-rich plasma and the signaling component is amniotic fluid.

5. The formulation of claim 1, wherein the first volume is greater than the second volume.

6. The formulation of claim 1, wherein the first volume is between 50% and 150% greater than the second volume.

7. The formulation of claim 1, wherein the activator component includes an anticoagulant.

8. A method for activating signaling molecules outside a native physiological response, the method comprising:
obtaining a formulation that includes a first volume of an activator component and a second volume of a signaling component; and
injecting or applying the formulation to a treatment area of a patient.

9. The method of claim 8, wherein injecting or applying the formulation to the treatment area of the patient comprises:
injecting the formulation subcutaneously into the patient's scalp for hair restoration.

10. The method of claim 8, wherein injecting or applying the formulation to the treatment area of the patient comprises:
injecting the formulation subcutaneously in or around the patient's face to repair damaged areas of skin.

11. The method of claim 8, wherein injecting or applying the formulation to the treatment area of the patient comprises:
applying the formulation to the patient's skin through microneedling.

12. The method of claim 8, wherein injecting or applying the formulation to the treatment area of the patient comprises:
injecting the formulation as a natural biological filler.

13. The method of claim 8, wherein injecting or applying the formulation to the treatment area of the patient comprises:
injecting the formulation intravaginally for vaginal rejuvenation.

14. The method of claim 8, wherein injecting or applying the formulation to the treatment area of the patient comprises:
injecting the formulation into the musculature of the patient's penis.

15. The method of claim 8, wherein injecting or applying the formulation to the treatment area of the patient comprises:
injecting the formulation into the patient's joint.

16. The method of claim 8, wherein the activator component is one of:
platelet-poor plasma;
platelet-rich plasma; or
platelet-rich fibrinogen.

17. The method of claim 16, wherein the signaling component is one of:
amniotic fluid; or
exosomes isolated from bone marrow or fat.

18. The method of claim 8, wherein the signaling component is one of:
amniotic fluid; or
exosomes isolated from bone marrow or fat.

19. The method of claim 8, wherein the first volume is greater than the second volume.

20. A formulation for activating signaling molecules outside a native physiological response comprising:
an activator component comprising one or more of platelet-poor plasma, platelet-rich plasma or platelet-rich fibrinogen; and
a signaling component comprising one or more of amniotic fluid or exosomes isolated from bone marrow or fat.
